(19) United States
(12) Patent Application Publication
(10) Pub. No.: US 2005/0015034 A1
(43) Pub. Date: Jan. 20, 2005

(54) TMC texas brace

(76) Inventors: Joseph Anthony Sansone, Houston, TX (US); Gordon Ryan Stelck, Houston, TX (US)

Correspondence Address: Attn: Joseph A. Sansone;TMC Orthopedic, Suite 150, 1000 South Loop West, Houston, TX, 77054, US

(21) Appl. No.: 10/769,607

(22) Filed: Oct. 4, 2004

Related U.S. Application Data
(60) Provisional application No. 60/444,800, filed on Feb. 4, 2003.

(51) Int. Cl. A61F 5/00

(52) U.S. Cl. 602/19

(57) ABSTRACT

Lumbo sacral orthosis (apparatus) the anterior and posterior portions of the brace consist of a medical grade ABS plastic. They are lined with Velcro laminate foam of ½ inch thickness which is self conforming to eliminate pressure points. The anterior plastic has three compression pull straps which attach to three D-ring straps on the posterior section. The anterior straps are threaded through the D-ring and allow the patients apply the appropriate tension to the apparatus. The six straps are attached to the plastic via brass oxide rivets.

This device is a traditional rigid lumbo sacral orthosis yet incorporates the benefits of flexible and conforming materials. The apparatus which is modular in design allowing for multiple adjustments as well as a fit to a wide variety of patients' sizes due to its conformity, and its and flexible nature. The orthosis provides the compression, support and the comfort liner system increases patient compliance therefore the patient will wear the orthosis and limit the potential for further injury.

### SUMMARY OF THE INVENTION

Is a Lumbar Sacral Orthosis (LSO); Lumbar Spinal Support with Thoracic Lumbar Sacral Orthosis Options.

The body of the brace consists of an anterior and posterior medical grade, Velcro laminate foam, ½″ in thickness, self conforming as not to create pressure points to boney prominences, cut to pattern size, all edges are securely sewn with a surger and are finished with a Poly-Star fabric Bias tape. Both anterior and posterior liners are completely removable from the rigid portions of the brace.

Attached to the anterior liner is a rigid thermo-moldable, ⅛″ in thickness, medical grade ABS plastic shell (Main anterior body), that covers all but an inch inside the perimeter of the anterior liner. The anterior portion of the LSO is designed to fit just above the Symphis Pubis and one inch under the Xyphoid Process, distally to proximally, to cover the proximal frontal plane of the torso wrapping around the proximal anterior Sagittal plane.

Attached to the posterior liner is a rigid thermo-moldable, ⅛″ in thickness, medical grade ABS plastic shell (main posterior rigid panel), that covers all but an inch inside the perimeter of the posterior liner. The posterior panel is designed to fit just above the Glutial fold to just at inferior Scapula, distally to proximally, to cover the proximal posterior Coronal plane of the torso wrapping around the posterior Transverse plane.

Both anterior and posterior liners have a series of ¾″ air holes through them to allow the brace to breathe as well as aid the practitioner in checking the depth of the fit against the patient's torso. The anterior and posterior plastic panels have a series of ¾″ air holes through them to allow the brace to breathe as well as aid the practitioner in checking the depth of the fit against the patient's torso.

Attached to the anterior plastic panel is a plurality of 1½″ compression pull straps made from Velcro hook and loop material backed by Dacron webbing for strength. The plurality of straps is attached to the left and right sagittal side of the anterior plastic panel in relation to the posterior main plastic panel

Attached to the posterior plastic panel is a plurality of 1½″ D-Ring straps that are made from Dacron webbing with a 1″ piece of polyethylene plastic insert, with a roller ring sewn to the end. The plurality of straps attached to the posterior median side of the posterior plastic panel in relation to the opposite side and in relation to the position of the corresponding anterior straps.

All of the anterior and posterior straps are attached to the respective main plastic bodies with solid brass oxide rivets.

The anterior straps are looped through the posterior D-Ring straps roller ring and are pulled back towards the anterior section to create hydrostatic lift therefore providing the needed immobilization of the lumbo sacral spine and are fastened to themselves b the Velcro hook and loop.

These straps have sewing at 3″ intervals that allow the excess of the straps to be cut after initial fitting as so not to fray.

On the base LSO are a series of 3/16″ holes drilled in the center of the main anterior plastic body in ½″ intervals for adjustability.

A Sternal pad kit may be added to the anterior plastic body with Chicago screws. The sternal bar is made from 3/16″×⅝″ aluminum round edge bar stock with 3/16 holes drilled in 1/32″ intervals for adjustability in height. Attached to the Proximal end of the sternal bar is a heart shaped ⅛″ thick×4″ tall×6½″ wide medical grade ABS plastic attached to a ¼″ closed cell foam to prevent pressure against the chest. The height of the top of the sternal bar should fit between the Xyphoid Process and the Sternal Notch.

A Pectoral pad kit may be added to the anterior plastic body with the same method as the Sternal pad kit. The adjustment holes and attachment screws are the same as the sternal bar kit. The nature of the materials of the Pectoral pad bar are the same as the sternal pad kit. The position of the height of the pectoral pads are the same as the sternal pad kit with the exception that the pectoral pad T-bar is adjustable in its own right from side to side at the Sagittal plane left to right.

### BRIEF DESCRIPTION OF THE DRAWINGS

FIG. 1 Posterior rigid panel with liner and plurality of Dacron D-Ring straps.

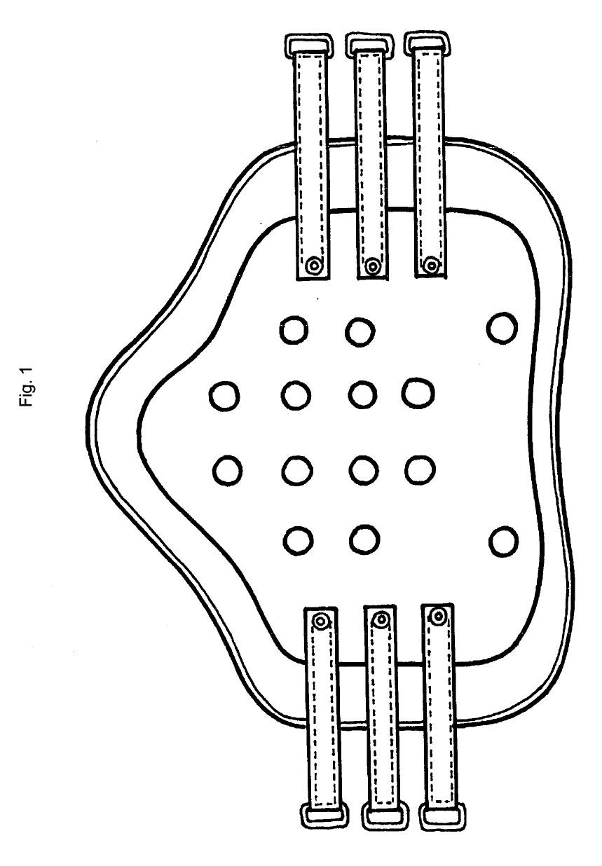

FIG. 2 Anterior rigid panel and liner with Dacron straps.

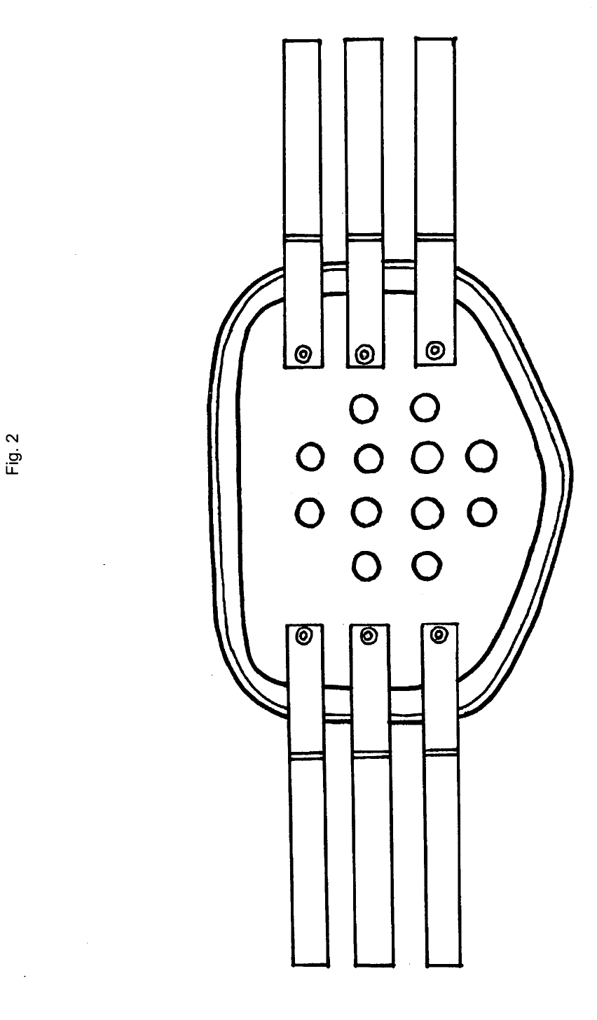

FIG. 3 Posterior Dacron D-Ring strap.

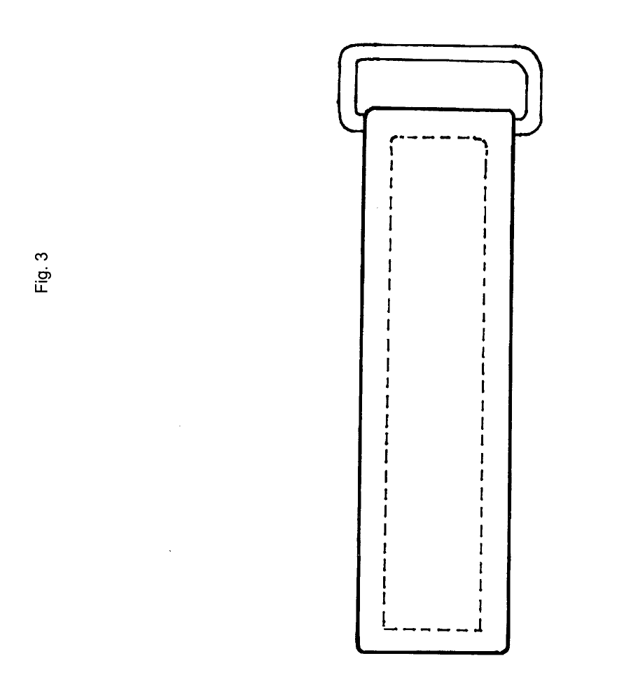

FIG. 4 Anterior Dacron strap.

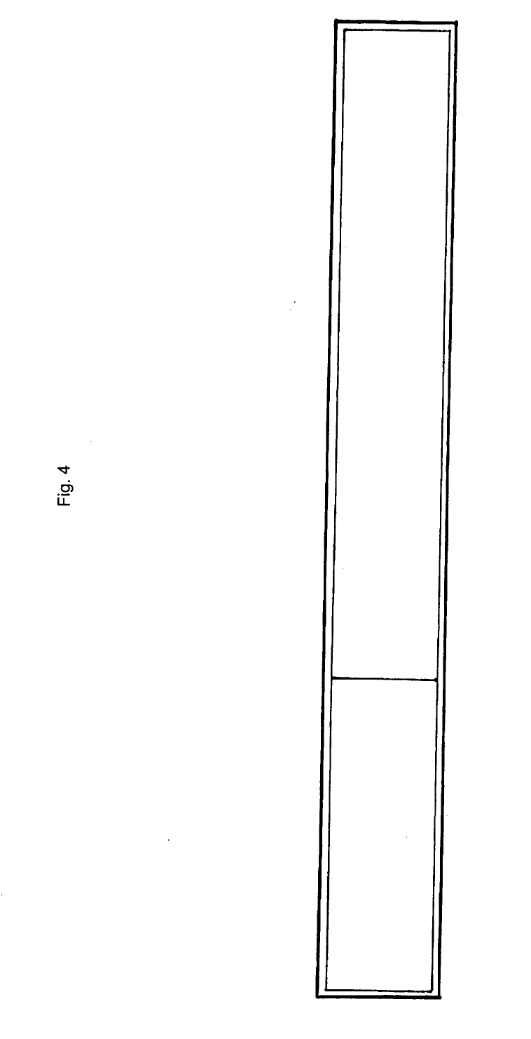

FIG. 5 Removable anterior liner with vent holes and surged edges.

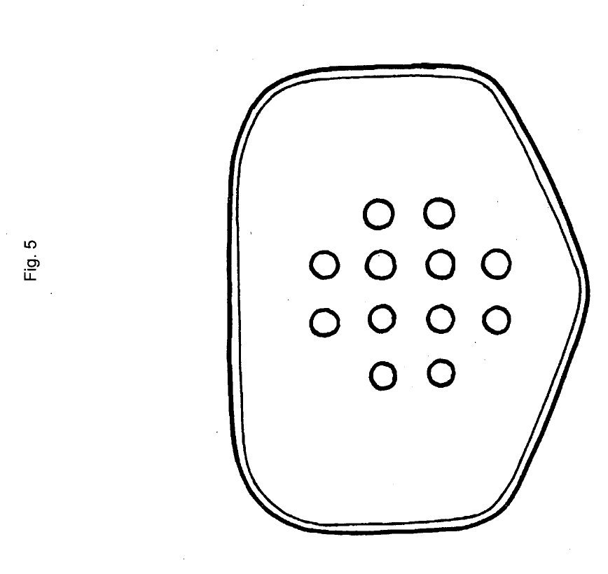

FIG. 6 Removable posterior liner with vent holes and surged edges.

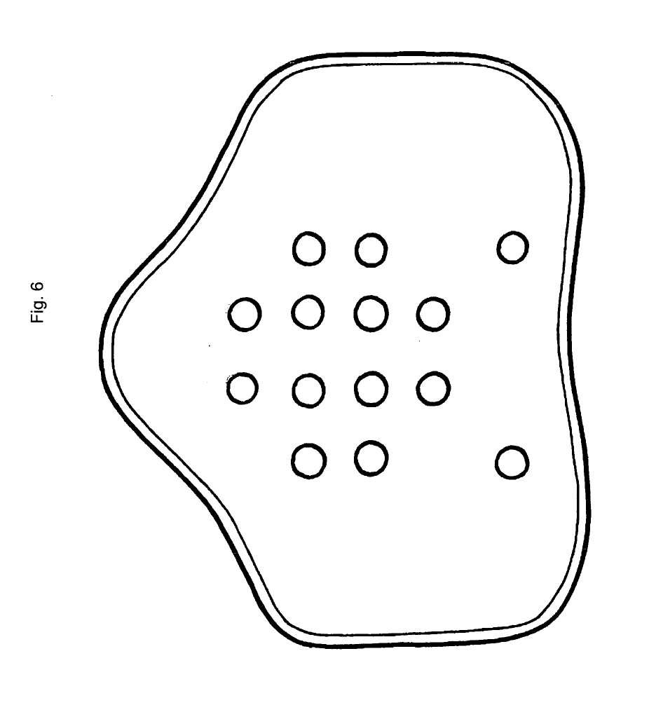

1. An orthosis comprising:
Lumbo sacral orthosis (apparatus) the anterior and posterior portions of the brace consist of a medical grade ABS plastic. They are lined with Velcro laminate foam of ½ inch thickness which is self conforming to eliminate pressure points. The anterior plastic has three compression pull straps which attach to three D-ring straps on the posterior section. The anterior straps are threaded through the D-ring and allow the patients apply the appropriate tension to the apparatus. The six straps are attached to the plastic via brass oxide rivets. The design of the LSO increases patient compliance due to the comfort and conforming nature of the liner and the simple strap system.
